(19) United States
(12) Patent Application Publication
(10) Pub. No.: US 2008/0145406 A1
(43) Pub. Date: Jun. 19, 2008

(54) DEVICES AND METHODS FOR OPHTHALMIC DRUG DELIVERY

(75) Inventors: Bahram Asgharian, Arlington, TX (US); Masood A. Chowhan, Arlington, TX (US)

Correspondence Address: ALCON, IP LEGAL, TB4-8, 6201 SOUTH FREEWAY, FORT WORTH, TX, 76134, US

(73) Assignee: Alcon Manufacturing Ltd., Fort Worth, TX (US)

(21) Appl. No.: 11/936,842

(22) Filed: Nov. 8, 2007

Related U.S. Application Data
(60) Provisional application No. 60/858,143, filed on Dec. 18, 2006.

(51) Int. Cl. A61F 2/00; A61K 31/57; A61K 31/70; A61K 39/395; A61M 35/00; A61P 27/02

(52) U.S. Cl. 424/427; 424/133.1; 514/179; 514/44; 604/294

(57) ABSTRACT

Disclosed are ophthalmic drug-delivery devices, comprising a body having a proximal end and a distal end, wherein the body includes a styrene elastomer matrix and a drug in contact with the matrix. Also disclosed are methods of treating or preventing an eye disease in a subject, that involve contacting an eye of the subject with an ophthalmic drug delivery device comprising a body having a proximal end and a distal end, wherein the body comprises a styrene elastomer matrix and a drug in contact with the matrix, wherein release of the drug from the device occurs over time following contacting of the device with the eye of the subject.

### BACKGROUND OF THE INVENTION

A. Field of the Invention

The present invention relates generally to the field of implantable drug-delivery devices and methods for the delivery of therapeutic agents. Particular drug-delivery devices of the invention are ophthalmic drug delivery devices that are comprised of a material that includes a styrene-based thermoplastic elastomeric polymer. Other particular aspects of the present invention pertain to the treatment of a disease of the posterior segment of the eye, such as choroidal neovascularization due to age-related macular degeneration.

B. Background of the Invention

The delivery of drugs to the eye presents a number of challenges to the clinician. Systemic administration of drugs for the treatment of diseases of the eye results in limited bioavailability of the drug at the site of disease because of the blood ocular barrier, made up by tights junctions of the retinal pigment epithelial cells and vascular endothelial cells. Although increasing the systemic dose of the drug may increase bioavailability within the eye, there is an associated risk of systemic toxicity which thus limits the use of systemic drugs.

Topical delivery of drugs to the eye often results in limited absorption of the drug into the eye due to the presence of the cornea and sclera. Furthermore, the blink mechanism results in removal of a substantial portion of topically applied drug, further limiting absorption. Although some delivery of the drug to the posterior segment may occur, it is often sub-therapeutic.

Intravitreal injection of drugs may result in effective delivery of a drug to the posterior segment. However, repeated injections are often necessary, which carry the risk of complications, including damage to the lens and infection within the eye.

Various drug delivery devices designed for delivery of therapeutic agents to the eye have been described. For example, U.S. Patent App. Pub. No. 20040219181 describes particular devices for intraocular delivery of drugs which include a drug core within a reservoir. U.S. Patent App. Pub. No. 20040133155 describes devices for intraocular implantation that include a nonlinear body portion that includes a lumen which can be refilled with a drug. It is unclear whether such devices result in improved bioavailability of agent to the posterior segment. Thermoplastic styrene elastomers are materials based on a co-polymer of styrene. This material has been used in the manufacture of pressure sensitive transdermal delivery systems (e.g., U.S. Patent App. Pub. No. 20040219198) and paclitaxel-elucing stents (TAXUS® Express2™, by Boston Scientific) but have not been described as ophthalmic drug delivery devices.

### SUMMARY OF THE INVENTION

The present invention provides for drug delivery devices that are composed of a styrene-based thermoplastic elastomeric polymer and an active agent that provide for controlled release of an active agent to a site in a subject. The drug delivery devices of the present invention have an advantage over bioerodable devices by providing for drug release over a longer period of time without the toxicity or inflammatory effects from bio-erosion byproducts, such as acids and alcohols. In general, the devices of the present invention can be easily manufactured using commercially available materials that are available in pure form and are very inexpensive. Further, styrene-based thermoplastic elastomeric polymer are known to be safe and acceptable for use as medical devices.

One embodiment of the present invention is directed to medical device that can be applied in the delivery of an active agent, such as a drug, to a site in a subject. For example, in particular embodiments the medical device includes a body configured to be inserted into a subject in the proximity of an eye of the subject, the body including a styrene elastomer matrix and a drug in contact with the matrix. Delivery can be to any part of the eye, but in particular embodiments the drug is delivered to the posterior segment of the eye. The “posterior segment” of the eye is defined to include the retina, choroid, retinal pigment epithelium, and vitreous.

A “styrene elastomer matrix” is a co-polymer matrix that incorporates styrene. The term “matrix” refers to the physical structure of the polymers of the present invention, which is addressed in greater detail below. The styrene elastomer matrix can include one or more copolymers selected from the group consisting of styrene-isoprene-styrene block copolymer (SIS), styrene-butadiene-styrene block copolymer (SBS), styrene-isoprene-butadiene-styrene block copolymer (SIBS), styrene-ethylene-butylene-styrene block copolymer (SEBS), and styrene-ethylene-propylene-styrene block copolymer (SEPS). In particular embodiments, the styrene elastomer matrix is SEBS. In certain embodiments, the drug or active agent is incorporated in the polymer matrix during manufacturing of the medical device.

The active agent can be any active agent known to those of ordinary skill in the art. For example, the active agent may be a drug selected from the group consisting of an anti-angiogenesis agent, an anti-glaucoma agent, an anti-infective agent, an anti-inflammatory agent, a growth factor, an immunosuppressant agent, and an anti-allergic agent. In particular embodiments, the active agent is an anti-angiogenesis agent that can be applied in the treatment of choroidal, subretinal, or retinal neovascularization of any cause. For example, the anti-angiogenesis agent may be anecortave acetate, 4,9(11)-pregnadien-17α,21-diol-3,20 dione, bevacizumab, ranibizumab, pegaptanib, or a receptor tyrosine kinase inhibitor (RTKi). Anti-angiogenesis agents are therapeutic agents that can be applied in the treatment of neovascularization, such as choroidal neovascularization associated with age-related macular degeneration.

The present invention is also generally directed to a method of treating or preventing a disease in a subject, comprising contacting the subject with a drug delivery device comprising a body configured to be inserted into the subject in a desired location, the body including a styrene elastomer matrix and a drug in contact with the matrix, wherein the drug is released from the device over time following the contacting. In particular embodiments, the method is a method of treating or preventing an eye disease in a subject that involves contacting an eye of the subject with an ophthalmic drug delivery device comprising a body configured to be inserted into the subject in the proximity of the eye, the body including a styrene elastomer matrix, and a drug in contact with the matrix, wherein the drug is released from the device over time following the contacting.

The styrene elastomer matrix can be any styrene elastomer matrix known to those of ordinary skill in the art. For example, the styrene elastomer matrix can be comprised of a copolymer selected from the group consisting of styrene-isoprene-styrene block copolymer (SIS), styrene-butadiene-styrene block copolymer (SBS), styrene-isoprene-butadiene-styrene block copolymer (SIBS), styrene-ethylene-butylene-styrene block copolymer (SEBS), and styrene-ethylene-propylene-styrene block copolymer (SEPS). In particular embodiments, the styrene elastomer matris is SIBS.

The term “subject” refers to either a human or non-human, such as primates, mammals, and vertebrates. In particular embodiments, the subject is a human. The eye disease to be treated or prevented includes any eye disease, with non-limiting examples including age-related macular degeneration, diabetic retinopathy, chronic glaucoma, retinal detachment, sickle cell retinopathy, retinal neovascularization, subretinal neovascularization; rubeosis irides, retinitis, choroiditis, posterior uveitis, neoplasms, retinoblastoma, pseudoglioma, neovascular glaucoma; neovascularization resulting following a combined vitrectomy and lensectomy, vascular diseases, retinal ischemia, choroidal vascular insufficiency, choroidal thrombosis, neovascularization of the optic nerve, diabetic macular edema, cystoid macular edema, macular edema, retinitis pigmentosa, retinal vein occlusion, proliferative vitreoretinopathy, angioid streaks, retinal artery occlusion, and neovascularization due to ocular injury. In particular embodiments, the eye disease is age-related macular degeneration, and the drug is anecortave acetate, 4,9(11)-pregnadien-17α,21-diol-3,20 dione, bevacizumab, ranibizumab, or pegaptanib.

Contacting the medical device with the eye of a subject can be by any method known to those of ordinary skill in the art. For example, the ocular device can be implanted into a juxtasceral location, in a subconjunctival and sub-Tenon location.

The term “about” or “approximately” are defined as being “close to” as understood by one of ordinary skill in the art, and in one non-limiting embodiment the terms are defined to be within 10%, preferably within 5%, more preferably within 1%, and most preferably within 0.5%.

The use of the word “a” or “an” when used in conjunction with the term “comprising” in the claims and/or the specification may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the examples, while indicating specific embodiments of the invention, are given by way of illustration only. Additionally, it is contemplated that changes and modifications within the spirit and scope of the invention will become apparent to those skilled in the art from this detailed description.

### BRIEF DESCRIPTION OF THE DRAWINGS

The following drawings form part of this specification and are included to further demonstrate certain non-limiting aspects of the present invention. The invention may be better understood by reference to one or more of these drawings in combination with the description of illustrated embodiments presented below.

FIG. 1 depicts a cross-sectional view of an eye.

FIG. 2A, FIG. 2B depicts styrenic block copolymers. FIG. 2A—general structure; FIG. 2B—types of elastomer mid-blocks.

FIG. 3 depicts the morphology of a styrenic block copolymer.

FIG. 4 depicts a perspective view of one of the medical devices of the present invention.

FIG. 5 is a perspective view of one of the medical devices of the present invention with a flange at the proximal end.

FIG. 6 is a cross-sectional view of an eye showing placement of the medical device of FIG. 5 following placement in a juxtascleral location.

### DESCRIPTION OF ILLUSTRATIVE EMBODIMENTS

Unless otherwise stated, all ingredient amounts presented as a percentage are in percent weight/weight terms (wt. %).

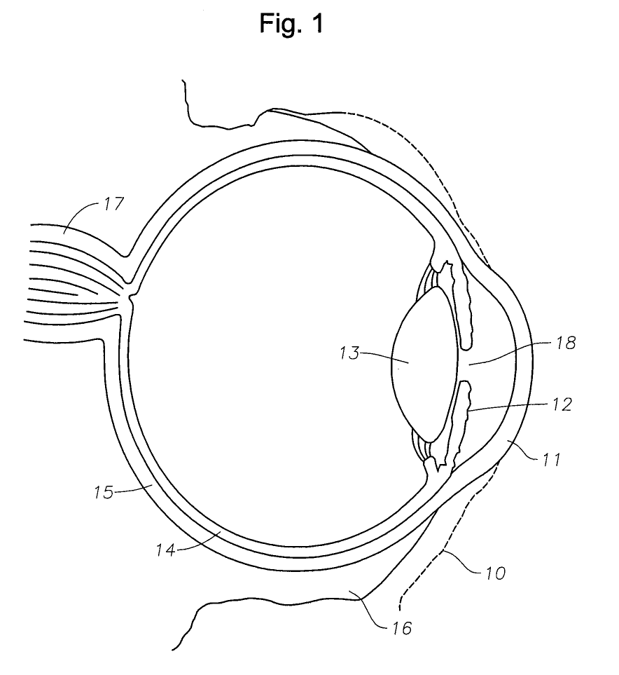

Diseases of the posterior segment of the eye are a significant cause of vision loss in the U.S. There are a number of vision-threatening disorders or diseases of the eye of a mammal that affect the posterior segment of the eye. A cross-section of an eye is diagrammatically represented in FIG. 1. Depicted is the conjunctiva 10, cornea 11, iris 12, lens 13, retina/choroid/retinal pigment epithelial layer 14, sclera 15, sub-Tenon's space 16, optic nerve 17, and pupil 18. Vision-threatening diseases that can affect the retina, retinal pigment epithelium, and choroid and include, for example, ocular neovascularization, ocular inflammation and retinal degenerations, such as age-related macular degeneration. Local sustained delivery of drugs to the posterior segment is crucial in the management of these diseases. Current methods of delivering therapeutic agents to the posterior segment of the eye are limited by the presence of the blood ocular barrier, lack of a sustained therapeutic effect, and risk of side effects with particular delivery modalities. Regarding drug-delivery devices, current devices are limited by toxicity and/or inflammation due to delivery matrix polymer or degradation products.

The present invention overcomes these deficiencies in the art by biomedical devices and materials that have the advantage of providing sustained drug release over a longer period of time with minimal toxicity or inflammation.

Styrene Elastomers

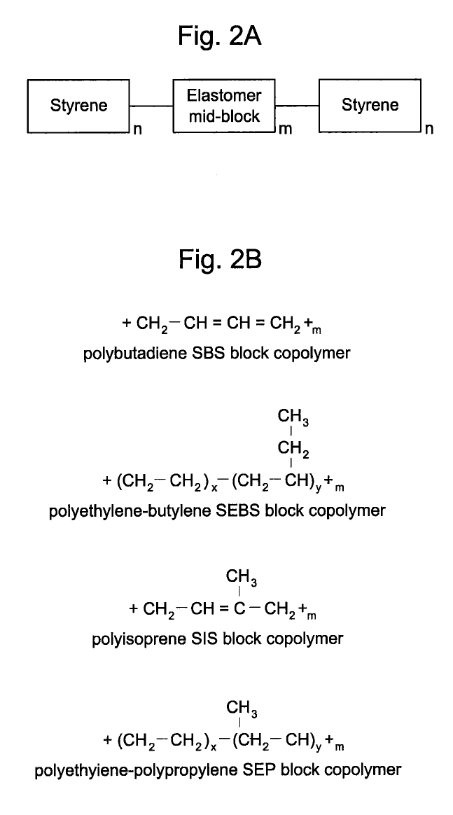

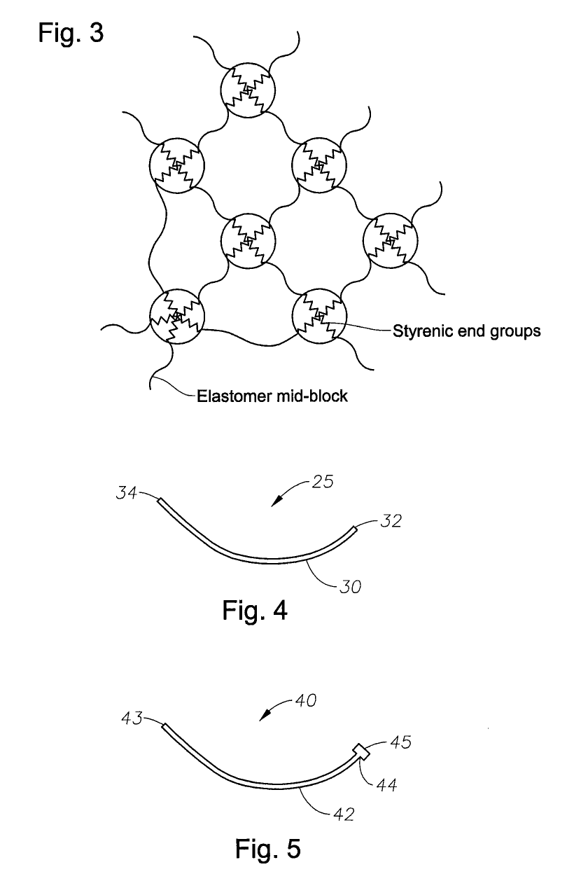

The styrene elastomers used in the present invention are copolymers composed of hard block (styrene) and soft block (butadiene, propylene, butylene, and/or a hydrogenation product thereof) polymers. FIG. 2A depicts the general structure of the styrene elastomers of the present invention, and FIG. 2B depicts examples of elastomer mid-blocks that can be included in the styrene elastomers of the present invention. FIG. 3 depicts the matrix morphology of a styrenic block copolymer.

Examples of styrene elastomers that can preferably be used in the present invention include SIS (styrene-isoprene-styrene block copolymer), SBS (styrene-butadiene-styrene block copolymer), SIBS (styrene-isoprene-butadiene-styrene block copolymer), SEBS (styrene-ethylene-butylene-styrene block copolymer), and SEPS (styrene-ethylene-propylene-styrene block copolymer.

Although styrene elastomers are not biodegradable, they are biocompatible and biostable and have been shown to have zero order release for a long period of time (Sipos et al., 2005).

Modifications or derivatives of styrene elastomers are contemplated as being useful with the methods and devices of the present invention. Derivatives may be prepared and such derivatives may be assayed for their desired properties by any method known to those of skill in the art.

In certain aspects, “derivative” refers to a chemically modified compound that still retains the desired effects of the compound prior to the chemical modification. Such derivatives may have the addition, removal, or substitution of one or more chemical moieties on the parent molecule. Non limiting examples of the types modifications that can be made to the compounds and structures disclosed throughout this document include the addition or removal of lower alkanes such as methyl, ethyl, propyl, or substituted lower alkanes such as hydroxymethyl or aminomethyl groups; carboxyl groups and carbonyl groups; hydroxyls; nitro, amino, amide, and azo groups; sulfate, sulfonate, sulfono, sulfhydryl, sulfonyl, sulfoxido, phosphate, phosphono, phosphoryl groups, and halide substituents. Additional modifications include the addition of a halide moiety to the styrene elastomer. Additional modifications can include an addition or a deletion of one or more atoms of the atomic framework.

The styrene elastomers used in the present invention can be synthesized by any method known to those of ordinary skill in the art. Alternatively, the styrene elastomers can be obtained from any of a number of commercial sources known to those of ordinary skill in the art. Exemplary commercially available styrene elastomers of such type include Krayton ®, Califlex (®; Shell Chemical), Tufprene®, Tuftek (®; Asahi Chemical Industry Co., Ltd.), Aron AR (Aron Chemical Industry Co., Ltd.), Rabalon (®; Mitsubishi Petrochemical Co., Ltd.), JSR-TR, JSR-SIS, Dynalon (Japan Synthetic Rubber Co., Ltd.), and Septon (Kuraray Co., Ltd.).

B. Medical Devices

Embodiments of the medical devices of the present invention are composed of a material that includes one or more styrene elastomers and one or more active agents.

The medical device materials of the present invention generally comprise a styrene elastomer in an amount of at least 50%, preferably at least 70%, and more preferably at least 80%. In some embodiments, the compositions comprise a styrene elastomer in an amount of at least 85%. In other embodiments, the compositions of the present invention comprise a styrene elastomer in an amount of at least 95%. In yet another embodiment, the compositions comprise a styrene elastomer in an amount of at least 99%.

Active agents include, but are not limited to, any component, compound, or small molecule that can be used to bring about a desired effect. Non-limiting examples of desired effects of the present invention include diagnostic and therapeutic effects. For example, a desired effect can include the diagnosis, cure, mitigation, treatment, or prevention of a disease or condition. An active agent can also affect the structure or function of body part or organ in a subject. In certain embodiments, the active agent is a drug, such as a hydrophobic drug. Active agents, discussed in greater detail in the specification below, can be obtained commercially from any of a number of sources, or can be chemically synthesized or obtained from natural sources.

Styrene elastomers are thermoplastic, and can be fabricated to a desired shape in heat molten gel state. In particular embodiments, the active agent is dispersed in polymer melt, which is then extruded to a desired shape. The active agent is dispersed within the matrix (see FIG. 3) of styrenic block copolymer. In particular embodiments, the active agent is non-covalently attached to the styrene elastomer. In certain embodiments, the shape is in accordance with existing ophthalmic drug delivery devices known to those of ordinary skill in the art (see, e.g., the devices set forth in U.S. Pat. No. 6,413,540 and U.S. Pat. No. 6,416,777, each incorporated by reference in its entirety). Additional examples are discussed in greater detail below.

In particular embodiments, the polymer and active agent are dissolved in a solvent, such as tetrahydrofuran, hexane, xylene, toluene, or similar organic solvents, or combinations of organic solvents. In some embodiments, the solvent is evaporated prior to melt extrusion.

In further embodiments, the active agent is mixed with the polymer, and the mixture of drug and polymer is coated onto a pre-formed device scaffold. The pre-formed device scaffold can be any device scaffold known to those of ordinary skill in the art, and include examples as set forth elsewhere in this specification. The preformed device scaffold may be made up of a polymer or other components known to those of ordinary skill in the art, such as those additional components discussed below. The pre-formed device may or may not be composed of a styrene elastomer.

Additional materials, such as other elastomers, triglyceride oils, or shape-memory materials can be added to the heat molten gel state to optimize the desired rigidity/flexibility of the device or the rate of drug release from the device. In particular, the composition can contain up to 30% of pharmaceutically acceptable oils, such as castor oil or a mixture of oils.

For example, in some embodiments, the medical device includes one or more additional elastomers, such as olefin elastomers. Olefin elastomers may comprise a copolymer of ethylene and propylene, or a copolymer further comprising third comonomer of alpha-olefin or diene. Exemplary commercially available olefin elastomers of such type include Milastomer, Tafmer (®; Mitsui Petrochemical Industries Co., Ltd.), Sumitomo TPE (Sumitomo Chemical Industries Co., Ltd.) and Thermorun (®; Mitsubishi Petrochemical Co., Ltd.).

Examples of shape memory materials include shape memory polyurethanes, crosslinked trans-polyoctylene rubber, polynorbornene polymers, nitinol, polyethylene, PMMA, polyurethane, cross-linked polyethylene, cross-linked polyisoprene, polycycloocetene, polycaprolactone, copolymers of (oligo)caprolactone, PLLA, PL/DLA copolymers, PLLA PGA copolymers, and other shape memory materials well-known to those of ordinary skill in the art.

Use of a styrene elastomer in the fabrication of a medical device has another merit that the finished device can be further shaped into the desired contour. For example, a medical device for implantation into a sub-tenon's location can be re-heated and bent into a desired contour, for example, immediately before the operation after examining the eye. In some embodiments, the device is sterilized by heat or gamma sterilization, if the drug is stable when exposed to gamma irradiation.

One of the present medical devices is shown in FIG. 4. Medical device 25 includes body 30, proximal end of body 32, and distal end of body 34. In particular embodiments, the body is comprised of a strand. The body may be non-linearly shaped, as with body 30. In other embodiments, the body is linearly shaped.

In the embodiment shown in FIG. 4, the strand is solid. Certain other embodiments include a channel through length of body 30 that allows for the passage of a guide wire to facilitate placement of medical device 25 in a desired location or insertion of a composition comprising one or more additional active agents.

Proximal end of body 32 and distal end of body 34 of device 25 are not tapered. In other embodiments, the body is tapered. The proximal end 32 and distal end 34 of body 30 may be rounded or blunt. In some embodiments, the proximal end and distal end are dissimilar. For example, the distal end may be broader and include a flattened configuration to allow for increased delivery of active agent to a site in a body.

On cross section (not shown), body 30 is rounded. In other embodiments, the body can be of any cross-sectional appearance, such as oval or rectangular. For example, body 30 may be flattened to allow for greater contact of the body of the device with the underlying sclera following placement.

Body 30 of device 25 is of a nonlinear shape, or curved. In other embodiments, the body of the medical device is straight. The medical device may be configured to a desired shape or configuration following manufacture, such as at the time of surgery, by heating the device and shaping it once the surgeon evaluated the patient immediately prior to implantation.

In the embodiment shown in FIG. 5, body 42 of device 40 includes a flange-shaped proximal end 45. In some embodiments, the flange-shaped proximal end serves as a handle, for holding the device or to stabilize it to allow for proper placement. In other embodiments, a flange attached to the proximal end of the body includes one or more holes for suturing a device to tissue to secure it to a particular location in a subject. For example, the body of the medical device may be a strand which includes a flange at the proximal end to allow for proper placement of the device and/or passage of suture to secure the device in a particular location.

In particular embodiments, the body of the medical device has a length of about 5 mm to about 40 mm. In more particular embodiments, the body of the medical device has a length of about 10 mm to about 30 mm. In some embodiments, the device is designed to be trimmed prior to implantation in a subject.

The diameter of the body of the medical device may be about 0.025 mm to about 5 mm. In particular embodiments, the diameter of the medical device is about 0.025 mm to about 1.5 mm.

C. Methods of Treating or Preventing a Disease

Certain embodiments of the present invention pertain to methods of treating or preventing a disease, such as an eye disease, in a subject that involves contacting an eye of a subject with one of the devices of the present invention, wherein the drug is released from the eye following the contacting.

Contacting the device with an eye of a subject can be by any method known to those of ordinary skill in the art.

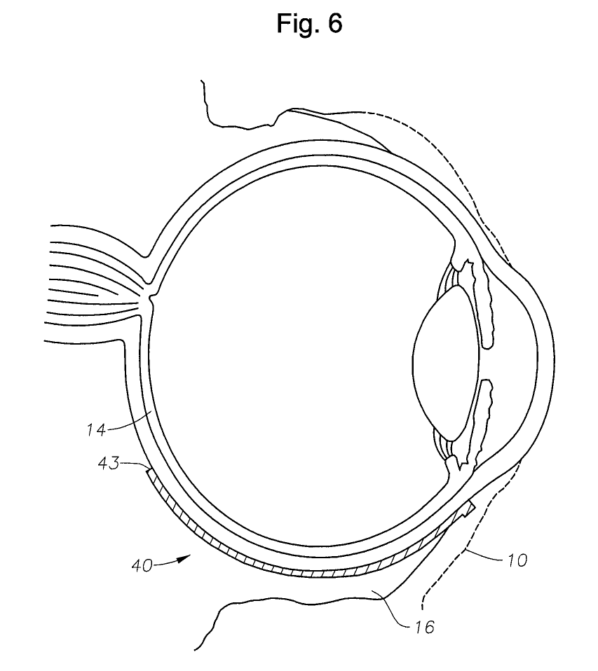

FIG. 6 is a cross-sectional diagram that demonstrates location of medical device 40 following placement of medical device 40 in an eye. Contacting and placement of device 40 in eye can be by any method known to those of ordinary skill in the art. For example, in some embodiments, a small conjunctival flap is created is created in conjunctiva 10, and the medical device is inserted beneath the flap and into sub-Tenon's space 16 such that distal end 43 of device 40 lies in a juxtascleral location that is sufficiently posterior to allow for sufficient delivery of active agent to the retina/choroid/retinal pigment epithelium 14, particularly in the region of the site of disease. The conjunctival flap may be closed with a resorbable suture. In some embodiments of the present methods, no conjunctival flap is required (i.e., the device is of a sufficiently small diameter such that it is passed directly through the conjunctiva and into proper location).

As noted above, the medical devices of the present invention are substantially non-biodegradable and inert. Thus, it is expected that the medical devices of the present invention can be left in place for a substantial period of time (e.g., days, weeks, or months). The device can be removed after a sufficient period of time, as determined by those of ordinary skill in the art.

In some of the methods set forth herein, repeat insertion of one or more additional devices is performed as part of a therapeutic regimen. Factors to consider in determining the need for repeat insertion of a device include the disease, the drug, and the configuration of the device.

In some embodiments, the methods set forth herein may include one or more secondary forms of therapy or prevention. For example, with regard to age-related macular degeneration, treatment with a secondary form of therapy, such as laser photocoagulation, may precede or follow implantation of a medical device of the present invention, such as a medical device that includes an active agent that is an anti-angiogenesis agent.

D. Active Agents

The drug-delivery devices of the present invention include one or more active agents in contact with the styrene elastomer matrix. Active agents include, but are not limited to, any component, compound, or small molecule that can be used to bring about a desired effect. Non-limiting examples of desired effects of the present invention include diagnostic and therapeutic effects. For example, a desired effect can include the diagnosis, cure, mitigation, treatment, or prevention of a disease or condition. An active agent can also affect the structure or function of body part or organ in a subject.

In certain embodiments, the active agent is a hydrophobic drug. A hydrophobic active agent includes an agent that is sparingly soluble in aqueous media (e.g., not completely dissolved in the media at the concentration at which it is administered in an aqueous composition). Thus, depending upon the use and concentration, an active agent may be considered water-insoluble in one situation but not water-insoluble in another situation. However, a person of ordinary skill in the art would recognize that the active agent does not need to be a hydrophobic drug in the context of the present invention. Typically, drug release increases as the drug content of the device increases. Drug release is also dependent on the hydrophobicity of the drug.

Ophthalmic Drugs

A preferred class of active agents includes ophthalmic drugs. In particular embodiments, the drugs are used to treat a disorder of the posterior segment. In more particular embodiments, the drug to treat a disorder of the posterior segment is a hydrophobic drug. For example, the drug may be anecortave acetate.

A preferred class of active agents includes ophthalmic drugs. Non-limiting examples include: anti-glaucoma agents, anti-angiogenesis agents; anti-infective agents; a anti-inflammatory agents; growth factors; immunosuppressant agents; and anti-allergic agents. Anti-glaucoma agents include beta-blockers, such as timolol, betaxolol, levobetaxolol, and carteolol; miotics, such as pilocarpine; carbonic anhydrase inhibitors, such as brinzolamide and dorzolamide; prostaglandins, such as travoprost, bimatoprost, and latanoprost; seretonergics; muscarinics; dopaminergic agonists; and adrenergic agonists, such as apraclonidine and brimonidine. Anti-angiogenesis agents include anecortave acetate (RETAANE™, Alcon™ Laboratories, Inc. of Fort Worth, Tex.) and receptor tyrosine kinase inhibitors. Anti-infective agents include quinolones, such as ciprofloxacin, moxifloxacin, and gatifloxacin, and aminoglycosides, such as tobramycin and gentamicin. Anti-inflammatory agents include non-steroidal and steroidal anti-inflammatory agents, such as suprofen, diclofenac, ketorolac, nepafenac, rimexolone, and tetrahydrocortisol. Growth factors include EGF. Anti-allergic agents include olopatadine and epinastine. The ophthalmic drug may be present in the form of a pharmaceutically acceptable salt, such as timolol maleate, brimonidine tartrate or sodium diclofenac.

In particular embodiments, the drug is a receptor tyrosine kinase (RTK) inhibitor, including any of those specific RTK inhibitors set forth above. Detailed information regarding RTK inhibitors is known and can be found in, for example, U.S. Patent App. Pub. No. 20060189608, hereby specifically incorporated by reference.

In other particular embodiments, the drug is a prostaglandin or a prostaglandin analog. For example, the prostaglandin analog may be latanoprost, bimatoprost, or travoprost.

In particular embodiments, the drug is a steroid. For example, the steroid may be a glucocorticoid, a progestin, a mineralocorticoid, or a corticosteroid. Exemplary coricosteroids include cortisone, hydrocortisone, prednisone, prednisolone, methylprednisone, triamcinolone, fluoromethalone, dexamethasone, medrysone, betamethasone, loteprednol, fluocinolone, flumethasone, or mometasone. Other examples of steroids include androgens, such as testosterone, methyltestosterone, or danazol. Often steroids are administered as ester, acetal, or ketal prodrugs, many of which are water-insoluble. These prodrugs are also considered to be steroids in the context of the present invention.

In particular embodiments, the drug is anecortave acetate. Anecortave acetate is an analog of cortisol acetate; among the modifications to the steroid are the removal of the 11β-hydroxyl group and an addition of a 21-acetate group. As a result of these modifications, anecortave acetate lacks the typical antiinflammatory and immunosuppressive properties of glucocorticoids. Anecortave acetate functions as an antiangiogenic agent, inhibiting blood vessel growth by decreasing extracellular protease expression and inhibiting endothelial cell migration. It is used in the treatment of neovascularization due to age-related macular degeneration.

Additional Active Agents

Although ophthalmic drugs are a preferred active agent of the present invention, the inventors contemplate that other active agents can be used. The following includes non-limiting examples of these other active agents, and it should be recognized that some these active agents may be generic to or identical to the ophthalmic drugs identified above. A reason for this is that some ophthalmic drugs can be used to treat or prevent other diseases or conditions. Further, it is also possible that some of the following active agents that are not identified in the above section can be used to treat ophthalmic diseases or conditions.

Active agents such as nucleic acids, proteins and peptides, hormones and steroids, chemotherapeutics, NSAIDs, vaccine components, analgesics, antibiotics, anti-depressants, etc. are contemplated as being useful in the context of the present invention. Non-limiting examples of nucleic acids that can be used include DNA, cDNA, RNA, iRNA, siRNA, anti-sense nucleic acid, peptide-nucleic acids, oligonucleotides, or nucleic acids that are modified to improve stability (e.g., phosphorothioates, aminophosphonates or methylphosphonates).

Proteins and peptides that can be used with the present invention include but are not limited to human growth hormone, bovine growth hormone, vascular endothelial growth factor, fibroblast growth factors, bone morphogenic protein, tumor necrosis factors, erythropoietin, thrombopoietin, tissue plasminogen activator and derivatives, insulin, monoclonal antibodies (e.g., anti-human epidermal growth factor receptor2 (Herceptin), anti-CD20 (Rituximab), anti-CD 18, anti-vascular endothelial growth factor, anti-IgE, anti-CD11a) and their derivatives, single-chain antibody fragments, human deoxyribonuclease I (domase alfa, Pulmozyme), type-1 interferon, granulocyte colony-stimulating factor, leuteinizing hormone releasing hormone inhibitor peptides, leuprolide acetate, endostatin, angiostatin, porcine factor VIII clotting factor, interferon alfacon-1, and pancrelipase (pancreatic enzymes).

Non-limiting examples of hormones and steroids that can be used include norethindrone acetate, ethinyl estradiol, progesterone, estrogen, testosterone, prednisone and the like. Other examples of steroids include glucocorticoids, progestins, mineralocorticoids, and corticosteroids. Exemplary corticosteroids include cortisone, hydrocortisone, prednisone, prednisolone, methylprednisone, triamcinolone, fluoromethalone, dexamethasone, medrysone, betamethasone, loteprednol, fluocinolone, flumethasone, or mometasone. Other examples of steroids include androgens, such as testosterone, methyltestosterone, or danazol. Often steroids are administered as ester, acetal, or ketal prodrugs, many of which are water-insoluble. These prodrugs are also considered to be steroids in the context of the present invention.

Chemotherapeutics that can be used include but are not limited to taxol (Paclitaxel), vinblastine, cisplatin, carboplatin, tamoxifen and the like.

Non-limiting examples of NSAIDs include piroxicam, aspirin, salsalate (Amigesic), diflunisal (Dolobid), ibuprofen (Motrin), ketoprofen (Orudis), nabumetone (Relafen), piroxicam (Feldene), naproxen (Aleve, Naprosyn), diclofenac (Voltaren), indomethacin (Indocin), sulindac (Clinoril), tolmetin (Tolectin), etodolac (Lodine), ketorolac (Toradol), oxaprozin (Daypro), and celecoxib (Celebrex).

Antibiotics include but are not limited to amoxicillin, penicillin, sulfa drugs, erythromycin, streptomycin, tetracycline, clarithromycin, tobramycin, ciprofloxacin, terconazole, azithromycin and the like.

Non-limiting examples of additional active ingredients can be found in Physician's Desk Reference 2000, 54th Edition, ISBN: 1563633302, AHFS 99 Drug Information, and Amer. Soc. of Health System, ISBN: 1879907917, which are incorporated by reference.

In some embodiments of the present methods, the devices of the present invention are designed for juxtascleral application. In other embodiments, the devices are placed in a subconjunctival location, a periocular location, a subtenon location, an intravitreal location, an intraocular location, or a subretinal location.

E. Diseases to be Treated

A “disease” or “health-related condition” can be any pathological condition of a body part, organ, or system of a subject. In certain instances, the condition can be the result of any cause, including for example, infection, genetic defect, and/or environmental stress. The cause may or may not be known.

“Treatment” and “treating” refer to administration or application of a therapeutic agent to a subject or performance of a procedure or modality on a subject for the purpose of obtaining a therapeutic benefit of a disease or health-related condition.

The term “therapeutic benefit” or “therapeutically effective” as used throughout this application refers to anything that promotes or enhances the well-being of the subject with respect to the medical treatment of his condition. This includes, but is not limited to, a reduction in the frequency or severity of the signs or symptoms of a disease.

“Prevention” and “preventing” are used according to their ordinary and plain meaning to mean “acting before” or such an act. In the context of a particular disease or health-related condition, those terms refer to administration or application of an agent, drug, or remedy to a subject or performance of a procedure or modality on a subject for the purpose of blocking the onset of a disease or health-related condition.

There are a number of vision-threatening disorders or diseases of the eye of a mammal including, but not limited to diseases of the retina, retinal pigment epithelium (RPE) and choroid. Such vision threatening diseases include, for example, ocular neovascularization, ocular inflammation and retinal degenerations. Specific examples of these disease states include diabetic retinopathy, chronic glaucoma, retinal detachment, macular edema, sickle cell retinopathy, age-related macular degeneration, retinal neovascularization, subretinal neovascularization, choroidal neovascularization, rubeosis irides, inflammatory diseases, chronic posterior and pan uveitis, neoplasms, retinoblastoma, pseudoglioma, neovascular glaucoma; neovascularization resulting following a combined vitrectomy and lensectomy, vascular diseases, retinal ischemia, choroidal vascular insufficiency, choroidal thrombosis, neovascularization of the optic nerve, diabetic macular edema, cystoid macular edema, macular edema, retinitis pigmentosa, retinal vein occlusion, proliferative vitreoretinopathy, angioid streak, and retinal artery occlusion, and, neovascularization due to penetration of the eye or ocular injury.

It is contemplated that the devices and methods of the present invention can be applied in the treatment of diseases that affect other parts of the eye, such as dry eye, meibomitis, glaucoma, conjunctivitis (e.g., allergic conjunctivitis, vernal conjunctivitis, giant papillary conjunctivitis, atopic keratoconjunctivitis), and iritis.

In additional embodiments of the invention, methods include identifying a patient in need of treatment. A patient may be identified, for example, based on taking a patient history, or based on findings on clinical examination

In order to increase the effectiveness of a treatment with one of the medical devices set forth herein, it may be desirable to combine these compositions with other therapies effective in the treatment of a particular disease or condition. Treatment using the devices of the present invention, for example, can precede or follow the other agent treatment by intervals ranging from minutes to weeks. It is contemplated that one may administer both modalities within about 12-24 h of each other and, more preferably, within about 6-12 h of each other. In some situations, it may be desirable to extend the time period for treatment significantly, where several days (2, 3, 4, 5, 6 or 7), several weeks (1, 2, 3, 4, 5, 6, 7 or 8) or even several months (1, 2, 3, 4, 5, 6, or more) lapse between the respective treatments.

F. Concentration of Active Agent

One embodiment of this invention includes methods of treating or preventing a disease or health-related condition that affects the eye of a subject that involves contacting the eye of the subject with an ophthalmic drug delivery device of the present invention, wherein the device is comprised of a styrene elastomer matrix and a drug in contact with the matrix, wherein release of the drug from the device occurs over time following contacting of the device with the eye of the subject.

The concentration of active agent that is combined with the styrene elastomer in the fabrication of the devices of the present invention is dependent on a number of factors, including the device size, shape, and nature of the drug. Any such concentration is contemplated in the manufacture of the devices of the present invention. As used herein, “concentration of active agent” refers to the percent weight of the active agent relative to the weight of all constituents used in the fabrication of the medical devices set forth herein, including the styrene elastomer and any additional components.

For example, the devices of the present invention may comprise at least about 0.001%, by weight, of an active ingredient. In other embodiments, the active ingredient may comprise between about 0.002% to about 50% of the weight of the compositions, and any range derivable therein. In still other embodiments, the active ingredient may comprise between about 0.5% to about 5% of the compositions. In further embodiments, the concentration of active agent is about 5% to about 30%. In still further embodiments, the concentration of active agent in the device is about 10% to about 20% by weight.

“Therapeutically effective amounts” are those amounts effective to produce beneficial results in the recipient. Such amounts may be initially determined by reviewing the published literature, by conducting in vitro tests or by conducting metabolic studies in healthy experimental animals. Before use in a clinical setting, it may be beneficial to conduct confirmatory studies in an animal model, preferably a widely accepted animal model of the particular disease to be treated. Preferred animal models for use in certain embodiments are rodent models, which are preferred because they are economical to use and, particularly, because the results gained are widely accepted as predictive of clinical value.

The actual dosage amount of an active agent, such as a drug, by the devices of the present invention can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

The device should be stable under the conditions of manufacture and storage. Sterilization following fabrication can be by any method known to those of ordinary skill in the art. For example, in some embodiments, sterilization is by gamma irradiation. The method selected will generally depend on various characteristics, such as the properties of any active agent or agents that are incorporated into the co-polymer matrix.

G. Controlled Release

In certain embodiments of the present invention, the medical device is designed to controllably or sustainably release the active agent to a target site. The phrases “controlled release”, “sustained release”, and similar terms and phrases describe a mode of active agent delivery that occurs when the active agent is released from the delivery device at an ascertainable and controllable rate over a period of time, rather than dispersed immediately upon application or injection.

Controlled or sustained release may extend for hours, days, months, or years and can vary as a function of numerous factors. For instance, the rate of release can depend on the type of styrene polymer in the matrix, and the configuration of the medical device.

H. Kits

In further embodiments of the invention, there is a provided a kit. The kit can include, in non-limiting aspects, a medical device of the present invention in a suitable container and instructions for insertion/placement. Containers of the kits can include a package or compartment. The container can include indicia on its surface. The indicia, for example, can be a word, a phrase, an abbreviation, a picture, or a symbol.

A kit can also include instructions for employing the kit components. Instructions may include variations that can be implemented. For example, the instructions may include information regarding placement and positioning of the medical device and information regarding the active agent. In some embodiments, the kit includes more than one medical device. In further embodiments, the kit includes a guidewire to facilitate proper positioning of the medical device in a juxtascleral location.

### EXAMPLES

The following examples are included to demonstrate certain non-limiting aspects of the invention. It should be appreciated by those of skill in the art that the techniques disclosed in the examples represent techniques discovered by the inventor to function well in the practice of the invention. However, those of skill in the art should, in light of the present disclosure, appreciate that many changes can be made in the specific embodiments which are disclosed and still obtain a like or similar result without departing from the spirit and scope of the invention.

### Example 1

### Processing of Medical Devices

The thermoplastic copolymers can be processed by standard processing techniques known to those of ordinary skill in the art. Examples of such techniques include injection molding, blow molding, spinning, vacuum forming, extrusion into tubes, extrusion into rods, extrusion into fibers, and/or extrusion into sheets. Devices can be made using solvent-based techniques where the polymer is dissolved in a solvent and then the drug is added, assuming the drug is also soluble in the solvent, and cast into the desired geometry by solvent elimination. Solvent-based systems where the drug matrix is the coating of the device are particularly preferred. The devices of the present invention can be sterilized by conventional methods, such as gamma sterilization, heat sterilization, or sterile filtration of the polymer melt.

The present medical devices methods can be made, used, and practiced without undue experimentation in light of the disclosure. The medical devices described above need not be made in the exact disclosed forms, or combined in the exact disclosed configurations to fall within the scope of the claims and their equivalents. Instead, it is possible to make substitutions, modifications, additions and/or rearrangements of the features disclosed above without deviating from their scope, which is defined by the claims and their equivalents. For example, the flange 45 of medical device 40 may include one or more suture holes to provide for suture placement to secure one of the devices of the present invention to a desired location.

The appended claims are not to be interpreted as including means-plus-function limitations, unless such a limitation is explicitly recited in a given claim using the phrase(s) “means for’ and/or “step for,” respectively.

### REFERENCES

The following references, to the extent that they provide exemplary procedural or other details supplementary to those set forth in this specification, are specifically incorporated by reference.
U.S. Pat. No. 6,413,540
U.S. Pat. No. 6,416,777
U.S. Pat. No. 6,995,186
U.S. Patent Publn. 2003/0055102
U.S. Patent Publn. 2004/0133155
U.S. Patent Publn. 2004/0219181
U.S. Patent Publn. 2004/0219198
U.S. Patent Publn. 2005/0158387
U.S. Patent Publn. 2006/0189608
AHFS 99 Drug Information
Amer. Soc. of Health System, ISBN: 1879907917
Physician's Desk Reference, 54^{th} Ed., ISBN: 1563633302, 2000.
Sipos et al., Biomacromolecules, 6(5):2570-2582, 2005.

1. An ophthalmic drug-delivery device comprising:
a body configured to be inserted into a subject in the proximity of an eye of the subject, the body including a styrene elastomer matrix; and
a drug in contact with the matrix.

2. The device of claim 1, wherein the body includes a linearly-shaped portion.

3. The device of claim 1, wherein the body has a non-linear shape.

4. The device of claim 1, wherein the body includes a flange-shaped proximal end.

5. The device of claim 4, wherein the flange-shaped proximal end includes one or more holes for suturing the device to the eye.

6. The device of claim 1, wherein the body has a length of about 5 mm to about 40 mm.

7. The device of claim 5, wherein the body has a length of about 10 mm to about 30 mm.

8. The device of claim 6, wherein the body has a diameter of about 0.1 mm to about 5 mm.

9. The device of claim 1, wherein the styrene elastomer matrix comprises a copolymer selected from the group consisting of styrene-isoprene-styrene block copolymer (SIS), styrene-butadiene-styrene block copolymer (SBS), styrene-isoprene-butadiene-styrene block copolymer (SIBS), styrene-ethylene-butylene-styrene block copolymer (SEBS), and styrene-ethylene-propylene-styrene block copolymer (SEPS).

10. The device of claim 9, wherein the styrene elastomer matrix is SIBS.

11. The device of claim 1, wherein the drug is selected from the group consisting of an anti-angiogenesis agent, an anti-glaucoma agent, an anti-infective agent, a nonsteroidal anti-inflammatory agent, a growth factor, an immunosuppressant agent, and an anti-allergic agent.

12. The device of claim 11, wherein the active agent is an anti-angiogenesis agent.

13. The device of claim 12, wherein the anti-angiogenesis agent is anecortave acetate, 4,9(11)-pregnadien-17α,21-diol-3,20 dione, bevacizumab, ranibizumab, pegaptanib, or a receptor tyrosine kinase inhibitor (RTKi).

14. A method of treating or preventing an eye disease in a subject, comprising:
contacting an eye of the subject with an ophthalmic drug delivery device comprising:
a body configured to be inserted into the subject in the proximity of the eye, the body including a styrene elastomer matrix; and
a drug in contact with the matrix;
wherein the drug is released from the device over time following the contacting.

15. The method of claim 14, wherein the styrene elastomer matrix comprises a copolymer selected from the group consisting of styrene-isoprene-styrene block copolymer (SIS), styrene-butadiene-styrene block copolymer (SBS), styrene-isoprene-butadiene-styrene block copolymer (SIBS), styrene-ethylene-butylene-styrene block copolymer(SEBS), and styrene-ethylene-propylene-styrene block copolymer (SEPS).

16. The method of claim 14, wherein the subject is a human.

17. The method of claim 14, wherein the eye disease is selected from the group consisting of age-related macular degeneration, diabetic retinopathy, chronic glaucoma, retinal detachment, sickle cell retinopathy, retinal neovascularization, subretinal neovascularization; rubeosis irides, retinitis, choroiditis, posterior uveitis, neoplasms, retinoblastoma, pseudoglioma, neovascular glaucoma; neovascularization resulting following a combined vitrectomy and lensectomy, vascular diseases, retinal ischemia, choroidal vascular insufficiency, choroidal thrombosis, neovascularization of the optic nerve, diabetic macular edema, cystoid macular edema, macular edema, retinitis pigmentosa, retinal vein occlusion, proliferative vitreoretinopathy, angioid streaks, retinal artery occlusion, and neovascularization due to ocular injury.

18. The method of claim 14, wherein the contacting comprising implanting the device in a subconjunctival and sub-Tenon's location in the subject.

19. The method of claim 14, wherein the disease is age-related macular degeneration.

20. The method of claim 14, wherein the drug is anecortave acetate, 4,9(11)-pregnadien-17α,21-diol-3,20 dione, bevacizumab, ranibizumab, or pegaptanib.
